(19) United States
(12) Patent Application Publication
(10) Pub. No.: US 2024/0041970 A1
(43) Pub. Date: Feb. 8, 2024

(54) TRADITIONAL CHINESE HERBAL COMPOSITION FOR TREATING ALLERGIC CONJUNCTIVITIS AND/OR ALLERGIC RHINITIS

(71) Applicant: EYE HOSPITAL, CHINA ACADEMY OF CHINESE MEDICAL SCIENCES, Beijing (CN)

(72) Inventors: Jiantao SONG, Beijing (CN); Chao XU, Beijing (CN)

(73) Assignee: EYE HOSPITAL, CHINA ACADEMY OF CHINESE MEDICAL SCIENCES, Beijing (CN)

(21) Appl. No.: 18/266,608

(22) PCT Filed: Dec. 26, 2022
(86) PCT No.: PCT/CN2022/141888

§ 371 Date: Jun. 12, 2023

(30) Foreign Application Priority Data
Oct. 28, 2021 (CN) 202111264405.2

(51) Int. Cl. A61K 36/758; A61K 36/23; A61K 36/21; A61K 36/8964; A61K 36/238; A61K 35/64; A61K 36/28; A61P 37/08

(52) U.S. Cl. CPC A61K 36/758; A61K 36/23; A61K 36/21; A61K 36/8964; A61K 36/238; A61K 35/64; A61K 36/28; A61P 37/08; A61K 2236/15

(57) ABSTRACT

A traditional Chinese herbal composition for treating allergic conjunctivitis and/or allergic rhinitis is prepared from the following raw material herbals in a weight ratio: 3-7 parts of szechuan pepper, 7-13 parts of szechuan lovage rhizome, 7-13 parts of belvedere fruit, 10-15 parts of common anemarrhena rhizome, 7-13 parts of divaricate saposhnikovia root, 4-8 parts of periostracum cicadae, and 7-13 parts of great burdock achene. The traditional Chinese herbal composition has the effects of dispelling wind, clearing heat and relieving itching, and has significant effects on treating allergic conjunctivitis and allergic rhinitis.

### CROSS REFERENCE TO THE RELATED APPLICATIONS

This application is the national phase entry of International Application No. PCT/CN2022/141888, filed on Dec. 26, 2022, which is based upon and claims priority to Chinese Patent Application No. 202111264405.2, filed on Oct. 28, 2021, the entire contents of which are incorporated herein by reference.

### TECHNICAL FIELD

The present invention particularly relates to a traditional Chinese herbal composition for treating allergic conjunctivitis and/or allergic rhinitis.

### BACKGROUND

Allergic conjunctivitis is a common disease in ophthalmology, and it is easy to attack repeatedly. Clinically, after an ophthalmic examination is performed, allergic conjunctivitis is found in many patients with allergic rhinitis who suffer from sneezing, nose running and rhinocnesmus, and these patients are finally confirmed with allergic conjunctivitis and allergic rhinitis. Allergic conjunctivitis/rhinitis refers to a non-infectious inflammatory disease of the conjunctiva and the nasal mucosa, which is mainly IgE-mediated mediator (mainly histamine) release and involves a variety of immunocompetent cells, cytokines, and the like after an atopic individual is exposed to an allergen. Allergic conjunctivitis/rhinitis has a high clinical morbidity, and can develop in all ages. Allergic conjunctivitis/rhinitis is easy to attack repeatedly during spring and autumn, which will cause continuous pains to the patients if no specific therapy is adopted and the disease condition is not alleviated, and therefore the life quality of the patients is seriously affected.

There are a lot of typical patients who suffer from allergic conjunctivitis complicated with allergic rhinitis, and they usually need to visit two departments, i.e., ophthalmology and rhinology after the onset, which makes the treatment extremely inconvenient. At present, treatments in ophthalmology of allergic conjunctivitis complicated with allergic rhinitis are mainly based on topical eye drops for symptomatic treatment, and common medicines comprise olopatadine, sodium cromoglycate, hormone eye drops, and the like. Such treatments are targeted at inhibiting mast cell degranulation and inhibiting release of inflammatory mediators causing allergies in early stage, but most of the treatments only relieve but cannot eliminate symptoms caused by immune reactions, and the application of the hormone eye drops can easily cause corticosteroid glaucoma, which has a high morbidity particularly in children and young people, and even results in high intraocular pressure. Treatments in rhinology are mainly based on oral administration of medicines like antihistamines or in combination with local sprays, but most of the treatments only relieve symptoms and maintain a short-time curative effect, and the disease condition is easy to reoccur after the treatments are suspended. The administration of systemic antiallergic medicines can further cause side effects such as heart disease, drowsiness, somnolence, and hypodynamia, and most of the nasal sprays contain hormone medicines and are not suitable for long-term use.

So far, no traditional Chinese herbal formulation for treating allergic rhinoconjunctivitis and no traditional Chinese herbal formulation capable of simultaneously treating allergic conjunctivitis and/or allergic rhinitis have been officially on the market.

### SUMMARY

In order to solve the above problems, the present invention provides a traditional Chinese herbal composition for treating allergic conjunctivitis and/or allergic rhinitis, which is prepared from the following raw material herbals in a weight ratio:
3-7 parts of szechuan pepper, 7-13 parts of szechuan lovage rhizome, 7-13 parts of belvedere fruit, 10-15 parts of common anemarrhena rhizome, 7-13 parts of divaricate saposhnikovia root, 4-8 parts of periostracum cicadae, and 7-13 parts of great burdock achene.

Further, the traditional Chinese herbal composition is prepared from the following raw material herbals in a weight ratio:
3 parts of szechuan pepper, 10 parts of szechuan lovage rhizome, 10 parts of belvedere fruit, 12 parts of common anemarrhena rhizome, 10 parts of divaricate saposhnikovia root, 6 parts of periostracum cicadae, and 10 parts of great burdock achene.

Further, the traditional Chinese herbal composition is a formulation prepared by taking herbal powders of the raw material herbals or water or organic solvent extracts of the raw material herbals as active ingredients and adding pharmaceutically acceptable adjuvants.

Further, the formulation is an oral formulation.

Further, the oral formulation is a granule, a powder, a pill, or a solution.

The present invention further provides a preparation method for the foregoing traditional Chinese herbal composition, which comprises the following steps:
(1) weighing the raw material herbals according to the foregoing ratio; and
(2) grinding the raw material herbals into powders or preparing an extracting solution of the raw material herbals in water or an organic solvent, and adding pharmaceutically common adjuvants or auxiliary components to obtain the composition.

The present invention further provides use of the foregoing traditional Chinese herbal composition in preparing a medicine for treating allergic conjunctivitis.

The present invention further provides use of the foregoing traditional Chinese herbal composition in preparing a medicine for treating allergic rhinitis.

The present invention finally provides use of the foregoing traditional Chinese herbal composition in preparing a medicine for dispelling wind, clearing heat and relieving itching.

In the herbal composition of the present invention, great burdock achene and belvedere fruit are bitter-cold and function as monarch drugs, which can dispel wind, clear heat, remove dampness, and relieve itching; divaricate saposhnikovia root and periostracum cicadae function as minister drugs to assist the monarch drugs to eliminate wind and relieve itching, and can also improve eyesight; common anemarrhena rhizome cools blood and nourishes Yin, eliminates wind without hurting Yin, and relieves itching by cooling blood; szechuan lovage rhizome ascends to the head and face, and also eliminates wind; szechuan pepper functions as a conductant drug to restrict the cold and cool herbals in the prescription, and has a good itching-relieving effect; and the effects of dispelling wind, clearing heat, and relieving itching are achieved together with all the ingredients.

The present invention follows academic thoughts of being holistic and dialectic and treating different diseases with same method in the field of the traditional Chinese medicine, and solves diseases in two sites of body with one prescription (both allergic conjunctivitis and allergic rhinitis have the same pathogenesis that is type I allergic reaction), which brings greater treatment benefits for patients with allergic conjunctivitis and allergic rhinitis. The present invention has been proven via experiments to have a significant effect on treating allergic rhinitis and allergic conjunctivitis, and is worthy of clinical popularization and application.

It is apparent that various other modifications, substitutions, and alterations can be made without departing from the basic technical concepts of the present invention according to the above contents of the present invention and based on the common technical knowledge and common practice in the field.

The above content of the present invention will be further described in detail below through specific implementations in the form of examples. However, this should not be construed as limiting the scope of the above subject matter of the present invention to the following examples.

All the technologies realized based on the above contents of the present invention shall fall within the scope of the present invention.

### DETAILED DESCRIPTION OF THE EMBODIMENTS

The solution, the granule, the powder, and the pill prepared from the composition of the present invention have a mature preparation process, and can be prepared with reference to the conventional methods in the prior art.

### Example 1. Preparation of the Medicine of the Present Invention

The formula is as follows: 3 g of szechuan pepper, 10 g of szechuan lovage rhizome, 10 g of belvedere fruit, 12 g of common anemarrhena rhizome, 10 g of divaricate saposhnikovia root, 6 g of periostracum cicadae, and 10 g of great burdock achene.

Preparation Method:
1) weighing the above raw material herbals according to the ratio; and
2) mixing the raw material herbals, adding water to decoct twice (the water was added in an amount of about 15 times of the mixture and the mixture was decocted for half an hour each time), mixing, filtering, and concentrating the decoctions to obtain a solution.

### Example 2. Preparation of the Medicine of the Present Invention

The formula is as follows: 3 g of szechuan pepper, 7 g of szechuan lovage rhizome, 7 g of belvedere fruit, 10 g of common anemarrhena rhizome, 7 g of divaricate saposhnikovia root, 4 g of periostracum cicadae, and 7 g of great burdock achene.

Preparation Method:
1) weighing the above raw material herbals according to the ratio; and
2) mixing the raw material herbals, adding water to decoct twice (the water was added in an amount of 10 times of the mixture and the mixture was decocted for half an hour each time), mixing and filtering the decoctions to obtain a filtrate, concentrating the filtrate into a thick paste, mixing well, drying the paste under reduced pressure, pulverizing into fine powders, adding dextrin and mixing well to obtain a granule by wet granulation method.

### Example 3. Preparation of the Medicine of the Present Invention

The formula is as follows: 7 g of szechuan pepper, 13 g of szechuan lovage rhizome, 13 g of belvedere fruit, 15 g of common anemarrhena rhizome, 13 g of divaricate saposhnikovia root, 8 g of periostracum cicadae, and 13 g of great burdock achene.

Preparation Method:
1) weighing the above raw material herbals according to the ratio; and
2) mixing the raw material herbals, adding water to decoct twice (the water was added in an amount of 12 times of the mixture and the mixture was decocted for 1 hour each time), mixing and filtering the decoctions to obtain a filtrate, concentrating the filtrate into a cream extract, and making the cream extract into pills to obtain a pill.

The beneficial effects of the present invention are illustrated by Test Examples below.

### Test Example 1. Clinical Efficacy of the Present Invention

1. Data and Method

1.1 General Data

From May 10, 2021 to Aug. 23, 2021, 36 patients diagnosed with allergic conjunctivitis complicated and allergic rhinitis were collected from Eye Hospital, China Academy of Chinese Medical Sciences, and they were divided into 2 groups according to a random number table method with 20 cases in the treatment group and 16 cases in the control group. There was no statistically significant difference in aspects such as age, gender, and disease course between the patients in the two groups before treatment (P>0.05), and therefore the two groups were comparable.

1.2 Medicines and Methods of Administration:

The treatment group was orally administered with the water decoction prepared in Example 1 with one dose a day and for twice a day, and the control group was orally administered with 10 mg of cetirizine capsules once a day.

2. Evaluation Indexes:
1) Ocular and nasal symptoms and signs were standardly divided into 4 grades (0: none, 1: mild: 2: moderate: and 3: severe);
2) Systemic adverse reactions and local adverse reactions in the eyes and nose were recorded.

2.1 Ocular Symptoms

Eye itching: (1) none; (2) mild: the patient won't notice eye itching when busy at work, but can feel itching when free, or has intermittent eye itching, but it's not severe enough to need to be scratched with hand; (3) moderate: the patient has obvious eye itching, and can still tolerate this symptom, but has the desire to scratch it with hand; and (4) severe: the patient has very obvious and continuous eye itching and stabbing pain, and cannot tolerate these symptoms, and therefore normal life is affected.

Tearing: (1) none; (2) tears are subjectively perceived in the conjunctival sac but do not flow out of the eyelid margin; (3) tears exist in the conjunctival sac, and occasionally overflow the eyelid margin to the cheek; and (4) tears regularly or continuously overflow to the cheek;

2.2 Ocular Signs

Conjunctival congestion: (1) none; (2) mild: congestion takes place in palpebral conjunctiva and fornix in a reticular form; (3) moderate: congestion takes place in palpebral conjunctiva, fornix and bulbar conjunctiva in a reticular form; and (4) severe: congestion takes place in palpebral conjunctiva in a form of red patches, blood vessels are blurred, and obvious congestion takes place in fornix and bulbar conjunctiva;

Conjunctival edema: (1) none; (2) bulbar conjunctival edema can be seen under a slit lamp examination; (3) moderate; bulbar conjunctival edema can be directly seen under natural light; and (4) severe: bulbar conjunctival edema with vesicular protuberances;

Secretions: (1) none; (2) a small amount of mucous secretions, which are filamentous; (3) moderate: a moderate amount of secretions, which are linear; and (4) severe: an excessive amount of secretions, which are flaky or adhesive to eyelid tissues upon awakening in the morning.

2.4 Nasal Symptoms:

Rhinocnesmus: (1) none; (2) mild and intermittent; (3) moderate: an ant walking sensation that can be tolerated; and (4) severe: an ant walking sensation that cannot be tolerated.

Sneezing (number of consecutive sneezings): (1) none; (2) mild: 3-9; (3) moderate: 10-14; (4) severe: ≥15.

Nose running (number of nose blowings per day): (1) none; (2) mild: =4; (3) moderate: 5-9; (4) severe ≥10.

2.5 Nasal Signs

Conditions of the nasal mucosa, inferior turbinate, nasal floor and nasal septum are observed with a nasal speculum and divided into the following four grades.
(1) no swelling of the inferior turbinate; (2) mild: mild swelling of the inferior turbinate, and the middle turbinate and nasal septum can still be seen; (3) moderate: the inferior turbinate is tightly close to the nasal septum (nasal floor), and there is still a small gap between the inferior turbinate and the nasal floor (nasal septum); and (4) severe: the inferior turbinate, the nasal floor and the nasal septum are tightly close to one another, and the middle turbinate cannot be seen.

3. Evaluation Method

The effective rate evaluation method is based on standards established in Clinical Development Guidelines for Pharmaceutical Products for Treatment of Allergic Rhinoconjunctivitis published by the European Medicines Agency, Chinese Expert Consensus on Diagnosis and Treatment of Allergic Conjunctivitis by Chinese Journal of Ophthalmology, 2018, and Guidelines for Diagnosis and Treatment of Allergic Rhinitis (Chinese Journal of Otorhinolaryngology Head and Neck Surgery, 2016. Vol. 51), and is divided into 4 grades (0: none; 1: mild; 2: moderate; and 3: severe), and it is considered effective if two symptoms (or signs) are improved by one grade or more, or same symptom (or sign) is improved by two grades or more. ((2) to (1), (3) to (2) or (4) to (3) represents a one-grade improvement, and (3) to (1) represents a two-grade improvement).

4. Efficacy Evaluation Results.

The results are shown in Tables 1 and 2.

TABLE 1
Comparison of the ocular efficacies of the two groups

| | | | | Effective |
|---|---|---|---|---|
| | Groups | Effective | Ineffective | Rate |
| 1-week | Treatment | 18 | 3 | 85.7% |
| | Group | | | |
| treatment | Control | 11 | 5 | 68.75% |
| | Group | | | |

TABLE 2
Comparison of the nasal efficacies of the two groups

| | | | | Effective |
|---|---|---|---|---|
| | Groups | Effective | Ineffective | Rate |
| 1-week | Treatment | 17 | 3 | 85.0% |
| | Group | | | |
| treatment | Control | 12 | 4 | 75.0% |
| | Group | | | |

As can be seen from Tables 1-2: the effective rate of the herbal composition of the present invention for treating allergic conjunctivitis and allergic rhinitis reaches more than 85%, and compared with the western medicine cetirizine, the effective rate has been improved by 13%-19%, and no adverse reactions have been found.

### Test Example 2. Typical Case

Chen, female, 50 years old, complained of intolerable eye itching, eye reddening, and tearing lasting for one week, accompanied by sneezing, nose running, and rhinocnesmus. This condition took place every autumn. To figure out, she came to visit in Eye Hospital, China Academy of Chinese Medical Sciences. After examination on her, we found congestion in a form of red patches in palpebral conjunctiva, and congestion in a reticular form in fornix and bulbar conjunctiva, and she was diagnosed with allergic conjunctivitis complicated with allergic rhinitis. She was orally administered with the water decoction prepared in Example 1 with one dose a day and for twice a day. After two doses were taken, the symptoms were obviously alleviated, and eye itching and rhinocnesmus were greatly alleviated. After two weeks of treatment, no sneezing or rhinocnesmus was found, eye itching became mild, no congestion in bulbar conjunctiva was found, and congestion in palpebral conjunctive was alleviated. Continuing with 7 doses thereafter, no ocular or nasal symptoms were further found.

In conclusion, the composition of the present invention solves diseases in two sites of body with one prescription (namely, allergic conjunctivitis and allergic rhinitis which have the same pathogenesis that is type I allergic reaction), achieves significant effects. The composition of the present invention brings greater treatment benefits for patients with allergic conjunctivitis and allergic rhinitis, and meanwhile, the composition avoids adverse reactions possibly caused by chemical medicines, and therefore is worthy of clinical popularization and application.

What is claimed is:

1. A traditional Chinese herbal composition for treating allergic conjunctivitis and/or allergic rhinitis, wherein
the traditional Chinese herbal composition is prepared from the following raw material herbals in a weight ratio:
3 parts of szechuan pepper, 10 parts of szechuan lovage rhizome, 10 parts of belvedere fruit, 12 parts of common anemarrhena rhizome, 10 parts of divaricate saposhnikovia root, 6 parts of periostracum cicadae, and 10 parts of great burdock achene.

2. The traditional Chinese herbal composition according to claim 1, wherein the traditional Chinese herbal composition is a formulation prepared by taking herbal powders of the raw material herbals or water or organic solvent extracts of the raw material herbals as active ingredients and adding pharmaceutically acceptable adjuvants.

3. The traditional Chinese herbal composition according to claim 2, wherein the formulation is an oral formulation.

4. The traditional Chinese herbal composition according to claim 3, wherein the oral formulation is a granule, a powder, a pill, or a solution.

5. A preparation method for the traditional Chinese herbal composition according to claim 1, comprising the following steps:
(1) weighing the raw material herbals according to the weight ratio in claim 1; and
(2) grinding the raw material herbals into powders or preparing an extracting solution of the raw material herbals in water or an organic solvent, and adding pharmaceutically common adjuvants or auxiliary components to obtain the traditional Chinese herbal composition.

6. A use method of the traditional Chinese herbal composition according tom claim 1, comprising: using the traditional Chinese herbal composition in preparing a medicine for treating the allergic conjunctivitis.

7. A use method of the traditional Chinese herbal composition according to claim 1, comprising: using the traditional Chinese herbal composition in preparing a medicine for treating the allergic rhinitis.

8. A use method of the traditional Chinese herbal composition according to claim 1, comprising: using the traditional Chinese herbal composition in preparing a medicine for dispelling wind, clearing heat and relieving itching.

9. The preparation method according to claim 5, wherein the traditional Chinese herbal composition is a formulation prepared by taking herbal powders of the raw material herbals or water or organic solvent extracts of the raw material herbals as active ingredients and adding pharmaceutically acceptable adjuvants.

10. The preparation method according to claim 9, wherein the formulation is an oral formulation.

11. The preparation method according to claim 10, wherein the oral formulation is a granule, a powder, a pill, or a solution.

12. The use method according to claim 6, wherein the traditional Chinese herbal composition is a formulation prepared by taking herbal powders of the raw material herbals or water or organic solvent extracts of the raw material herbals as active ingredients and adding pharmaceutically acceptable adjuvants.

13. The use method according to claim 12, wherein the formulation is an oral formulation.

14. The use method according to claim 13, wherein the oral formulation is a granule, a powder, a pill, or a solution.

15. The use method according to claim 7, wherein the traditional Chinese herbal composition is a formulation prepared by taking herbal powders of the raw material herbals or water or organic solvent extracts of the raw material herbals as active ingredients and adding pharmaceutically acceptable adjuvants.

16. The use method according to claim 15, wherein the formulation is an oral formulation.

17. The use method according to claim 16, wherein the oral formulation is a granule, a powder, a pill, or a solution.

18. The use method according to claim 8, wherein the traditional Chinese herbal composition is a formulation prepared by taking herbal powders of the raw material herbals or water or organic solvent extracts of the raw material herbals as active ingredients and adding pharmaceutically acceptable adjuvants.

19. The use method according to claim 18, wherein the formulation is an oral formulation.

20. The use method according to claim 19, wherein the oral formulation is a granule, a powder, a pill, or a solution.
